(19) United States
(12) Patent Application Publication
(10) Pub. No.: US 2026/0096813 A1
(43) Pub. Date: Apr. 9, 2026

(54) KNOTLESS VALVE SUTURE WITH PREFORMED LOOP

(71) Applicant: Arthrex, Inc., Naples, FL (US)

(72) Inventors: Andrew C. Petry, Naples, FL (US); Derek C. Sullivan, Naples, FL (US)

(73) Assignee: Arthrex, Inc., Naples, FL (US)

(21) Appl. No.: 19/346,848

(22) Filed: Oct. 1, 2025

Related U.S. Application Data
(60) Provisional application No. 63/702,871, filed on Oct. 3, 2024.

(51) Int. Cl. A61B 17/04; A61B 17/06

(52) U.S. Cl. CPC A61B 17/0401; A61B 17/0487; A61B 17/06166; A61B 2017/0406; A61B 2017/0464; A61B 2017/0488

(57) ABSTRACT

A knotless valve suture assembly includes a first predefined suture loop, a first suture end coupled with the first predefined suture loop, a second predefined suture loop, and a second suture end coupled with the second predefined suture loop. At least one implant clip is included. At least one of the first and second predefined suture loops is configured to be tensioned over the at least one implant clip in response to the tensioning force applied to at least one of the first and second suture ends, respectively, to press the at least one implant clip against a replacement valve and the replacement valve against tissue.

### CROSS-REFERENCE TO RELATED APPLICATION

This application claims priority under 35 U.S.C. § 119(e) and the benefit of U.S. Provisional Application No. 63/702,871 entitled KNOTLESS VALVE SUTURE WITH PREFORMED LOOP, filed on Oct. 3, 2024, the entire disclosure of which is incorporated herein by reference.

### BACKGROUND

The present disclosure generally relates to a valve suture and, more particularly, to a knotless valve suture with one or more predefined loops for cardiovascular valve procedures. During cardiovascular procedures, such as valve repairs or valve replacements, sutures can be used to attach a valve to tissue. The sutures often form loops or stitches through a shuttling process. In various implementations, the disclosure provides for devices and methods that may assist in the operation of suture assemblies by having one or more predefined and tensionable loops.

### SUMMARY

The present disclosure generally relates to a knotless valve suture assembly. The suture assembly may include at least one preformed loop. In certain aspects, the suture assembly may form multiple tensioned stitches with a single, dual looped suture having two preformed loops and first ends and second ends, which may be tail ends. A first tail end and a first preformed loop may be coupled with a first needle, and a second tail end and a second preformed loop may be coupled with a second needle. The needles may carry portions of the suture through tissue and an implant, such as a replacement valve. A tensioning force may be applied to both ends to tension the preformed loops over an implant clip.

In additional or alternative configurations, the suture assembly may form separate tensioned stitches with multiple, single looped sutures. The single loop suture may have a preformed loop, a fixed end, and a tail end. A first needle can be coupled to the tail end, and a second needle can be coupled to the preformed loop to carry the suture portions through tissue and the implant. A tensioning force may be applied to the tail end to tension the preformed loop over an implant clip.

The suture assembly may also include the implant clip, which may be pressed into the implant by the tensioned sutures. One suture may engage a single implant clip, multiple sutures may engage a single implant clip, and/or a single suture may engage multiple implant clips. In valve replacement examples, predefined suture loops may be configured to press the implant clip against the replacement valve to assist in forming a seal between the tissue and the valve. The suture assembly can include at least one locking feature for engaging a respective suture. The locking feature can be configured to allow movement in a first direction for tensioning the sutures during the tensioning process and may be configured to lock or retain the sutures in a tensioned state over the respective implant clip.

These and other features, objects, and advantages of the present disclosure will become apparent upon reading the following description thereof together with reference to the accompanying drawings.

### BRIEF DESCRIPTION OF THE DRAWINGS

FIG. 1 is representative of a cardiovascular valve replacement with sutures to bring a replacement valve to engage an annulus, according to the present disclosure;

FIG. 2 is representative of an aortic annulus with suture assemblies arranged around the annulus, according to the present disclosure;

FIG. 3 is a schematic diagram of a suture assembly including a pledget a suture having two preformed loops and two ends, according to the present disclosure;

FIG. 4 is a partial schematic diagram of a suture assembly with a spliced segment and thickened portion forming a locking feature, according to the present disclosure;

FIG. 5 is a schematic diagram of a suture assembly extending through tissue and a replacement valve, where the suture assembly includes a pledget under the tissue, a suture, two needles, and an implant ring, according to the present disclosure;

FIG. 6 is a schematic diagram of a suture assembly extending through tissue and a replacement valve, where a tensioning force is applied to ends of the suture to tension preformed loops over an implant ring, according to the present disclosure;

FIG. 7 is a schematic diagram of a suture assembly extending through tissue and a replacement valve, where preformed loops of a suture are tensioned over an implant ring, according to the present disclosure.

FIG. 8 is a schematic diagram of a suture assembly extending through tissue and a replacement valve, where the suture assembly includes a pledget under the tissue, a suture, two needles, and a notched implant clip, according to the present disclosure;

FIG. 9 is a partial exploded view of a suture assembly extending through tissue and a replacement valve, where the suture assembly includes a suture and a folded pledget illustrated in an expanded state, according to the present disclosure;

FIG. 10 is a partial exploded view of a suture assembly extending through tissue and a replacement valve, where the suture assembly includes a suture and a folded pledget illustrated in a compressed state, according to the present disclosure;

FIG. 11 is a schematic diagram of a suture assembly including an annular pledget and multiple sutures with preformed loops, according to the present disclosure;

FIG. 12 is a flow diagram of a method of using a knotless suture assembly with a dual loop suture for a valve replacement, according to the present disclosure;

FIG. 13 is a schematic diagram of a suture assembly extending through tissue and a replacement valve, where the suture assembly includes a pledget under the tissue, a suture having a preformed loop and a tail end, a spliced segment, two needles, and an implant clip, according to the present disclosure;

FIG. 14 is a schematic diagram of a suture assembly extending through tissue and a replacement valve, where the suture assembly includes a pledget under the tissue, a suture having a preformed loop and a tail end, a grommet, and an implant clip, according to the present disclosure;

FIG. 15 is a schematic diagram of multiple suture assemblies having preformed loops and multiple implant clips to form an implant chain, according to the present disclosure;

FIG. 16 is a schematic diagram of a replacement valve with an implant ring formed from interlinked suture assemblies and C-shaped implant clips, according to the present disclosure;

FIG. 17 is a schematic diagram of a replacement valve with an implant ring formed from interlinked suture assemblies and S-shaped implant clips, according to the present disclosure; and

FIG. 18 is a flow diagram of a method of using a knotless suture assembly with a single loop suture for a valve replacement, according to the present disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

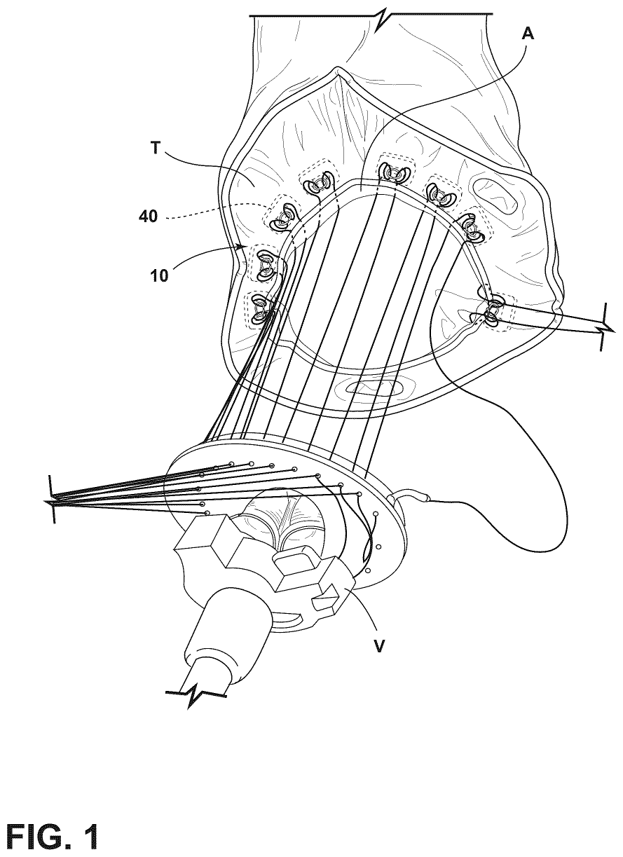

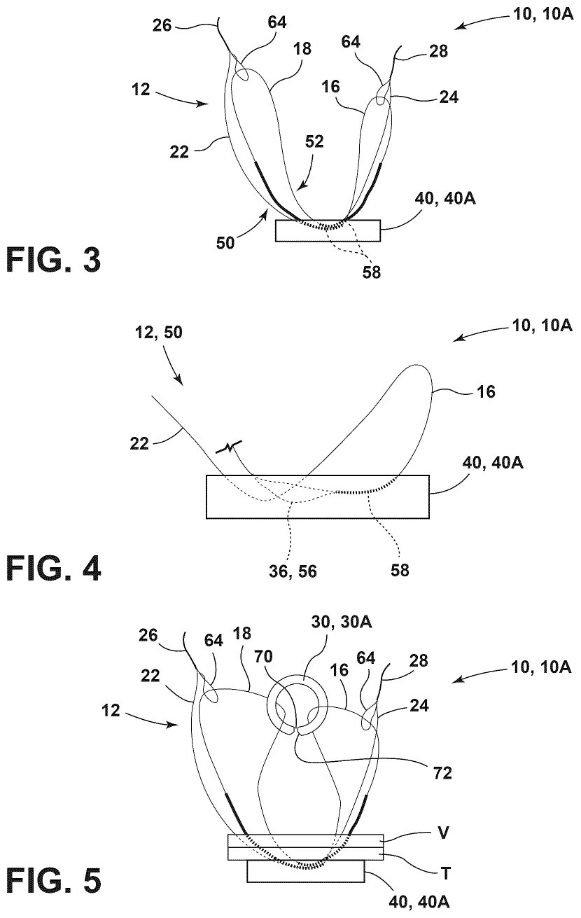

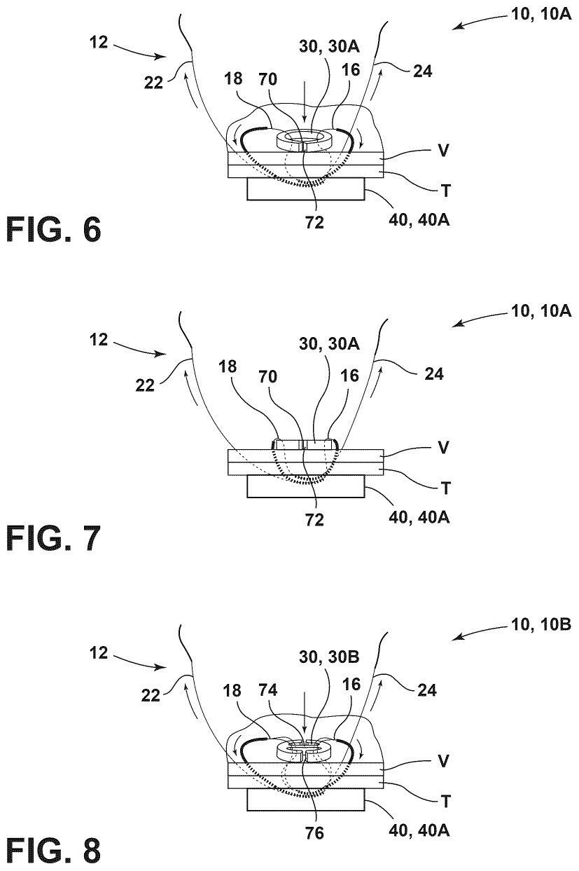

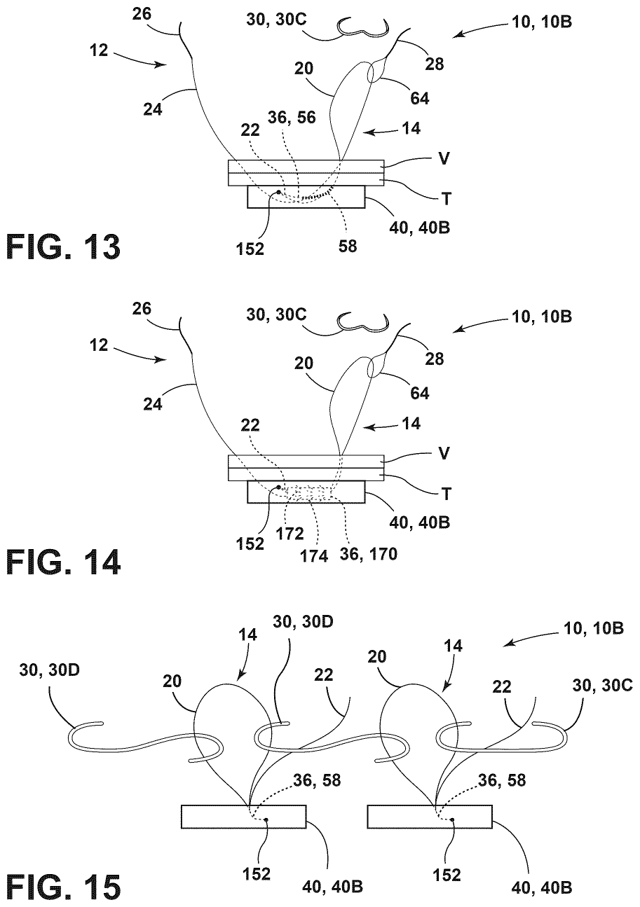

With reference to FIGS. 1-18, reference numeral 10 generally designates a knotless valve suture assembly, which may have multiple configurations illustrated herein as suture assemblies 10A, 10B and collectively referred to as the suture assemblies 10. The suture assembly 10A may form multiple tensioned stitches with a single, dual looped suture 12, as illustrated in FIGS. 3-11. The suture assembly 10B may form separate tensioned stitches with multiple single, looped sutures 14, as illustrated in FIGS. 13-17. The suture assembly 10 may include at least one predefined, tensionable loop, which may be two predefined or preformed loops 16, 18 with the dual loop suture 12 or a single predefined or preformed loop 20 with the single loop suture 14. Each suture 12, 14 may include a first suture end 22 and a second suture end 24. For the dual loop suture 12, the first and second ends 22, 24 may be tail ends, and the first suture end 22 may be coupled with the first predefined suture loop 16 and the second suture end 24 may be coupled with the second predefined suture loop 18. For the single loop suture 14, the first end 22 may be a fixed end while the second end 24 may be the tail end for adjusting the single predefined loop 20.

Two needles 26, 28 may be coupled with the suture assembly 10 for carrying the suture ends 22, 24 through tissue T and a replacement valve V. In the dual loop suture assembly 10A (see FIGS. 3-11), the predefined loops 16, 18 and the ends 22, 24 may be components of a single suture 12 and, therefore, may be integrally formed with one another. In such configurations, the first needle 26 may be operably coupled with the first end 22 and the second predefined loop 18, which can be arranged proximate to one another. The second needle 28 may be operably coupled with the second end 24 and the first predefined loop 16, which can be arranged proximate to one another. The needles 26, 28 may carry the ends 22, 24 and predefined loops 16, 18, respectively, through the tissue T and the valve V. In the single loop suture assembly 10B (see FIGS. 13-17), multiple sutures 14 can be used for providing the first and second loops 20. In such configurations, for each suture 14, the first needle 26 may be coupled with the tail end 24, and the second needle 28 may be coupled with the predefined loop 20.

The suture assembly 10 may also include an implant clip 30, which may have different configurations referred to herein as implant clips 30A-30E. The predefined suture loops 16, 18, 20 may be configured to be tensioned over at least one implant clip 30 in response to a tensioning force applied to one or both of the suture ends 22, 24, respectively. The predefined suture loops 16, 18, 20 may be configured to press the implant clip 30 against the replacement valve V to assist in forming a seal between the tissue T and the valve V. The suture assembly 10 typically can include at least one locking feature 36 for engaging a respective suture 12, 14. The sutures 12, 14 may generally be configured to extend through the locking feature 36, and the locking feature 36 can be configured to allow movement in a first direction for tensioning the sutures 12, 14 during the tensioning process. The locking feature 36 may generally be configured to reduce or prevent movement of the sutures 12, 14 in a second, opposing direction, forming a locking trap that may retain the sutures 12, 14 in a tensioned state over the respective implant clip 30.

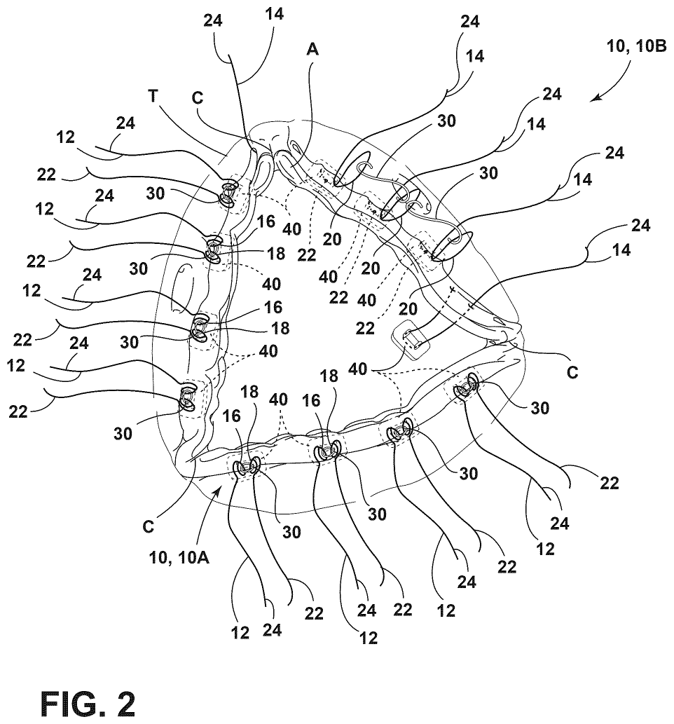

Referring to FIGS. 1 and 2, the suture assembly 10 may be configured for use with connecting devices and/or implants to soft tissue/muscle T. In non-limiting examples, the suture assembly 10 may be utilized for heart valve replacements or heart valve repairs. In such examples, the suture assembly 10 may be utilized for a mitral valve replacement or an aortic valve replacement. Using the aortic valve replacement as an example, an annulus A extends around leaflets of the aortic valve V. In the valve replacement, the leaflets are removed from a patient. The suture assembly 10 may be configured to draw the replacement valve V into the patient to implant and replace the valve V that was removed from the patient. Depending on the type of valve replacement, the valve replacement procedure may access the aortic valve via the aorta or the mitral valve via the septum.

The replacement valve V may be a mechanical valve V or may be a tissue-based valve V, which can often be a xenograft-type of tissue. The type of valve V may be chosen based on various factors, including patient age, patient diagnosis, prior health complications, medications, etc. The suture assembly 10 may be used with any type of replacement valve V without departing from the teachings herein. The suture assembly 10 may assist with defining a fluid-tight or blood-tight seal between the tissue T and the replacement valve V. Moreover, the suture assembly 10 may be re-tensioned over the implant clip(s) 30, as described herein, during the valve replacement procedure to maximize alignment of the replacement valve V and provide consistent pressure over the valve V for providing the fluid-tight seal.

Referring still to FIGS. 1 and 2, during the valve replacement procedure, the replacement valve V can be brought to engage and compress against the tissue T. The replacement valve V may generally be sized to correspond with a size of the annulus A or a tube into which the replacement valve V is being attached. For example, if the opening for the tube has a 27 mm diameter, a 27 mm replacement valve V may be used to reduce or prevent any gaps outside of the replacement valve V. Though specific exemplary sizes and proportions are described, it shall be understood that these serve as examples to aid understanding and are not limiting to the disclosure.

Multiple sutures 12, 14 and/or suture assemblies 10 may be used to secure the replacement valve V to the tissue T. For example, between about 12 to 17 sutures 12, 14 may be utilized in the procedure and arranged around the annulus A. The knotless aspect of the suture assembly 10 may be advantageous for more efficiently securing the replacement valve V to the tissue T without additional knots or crimping devices used with conventional sutures. Further, the implant clips 30 may assist in providing more even or more consistent pressure around the annulus A and, consequently, may provide a more consistent seal between the tissue T and the valve V.

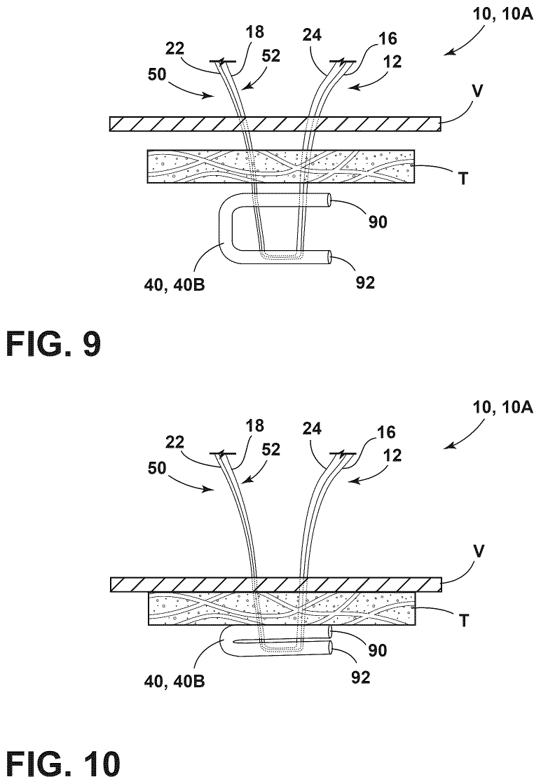

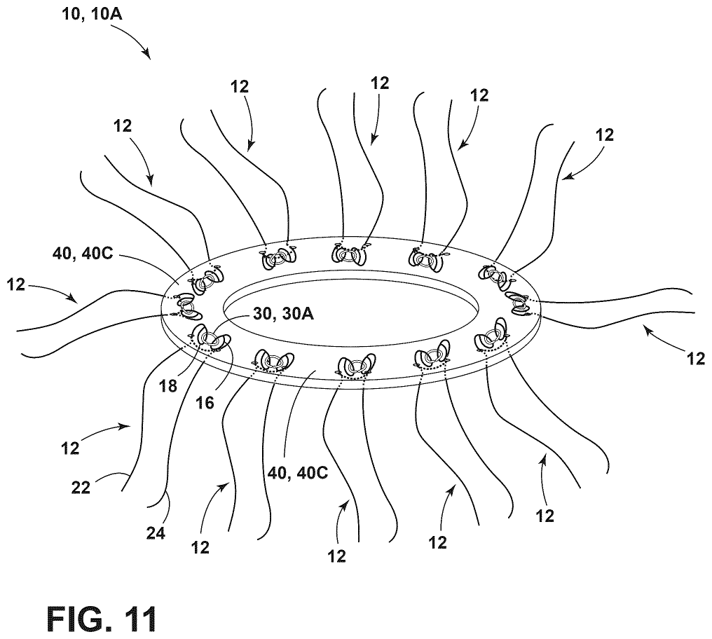

The suture assembly 10 may include at least one pledget 40 for supporting the suture(s) 12, 14. The pledget 40 may have a number of configurations, such as a rectangular or square profile referred to as a flat pledget 40A (see FIGS. 3-7 and 13-15), a tubular structure referred to as a folded pledget 40B (see FIGS. 9 and 10), and/or an annular or ring shape referred to as an annular pledget 40C (see FIG. 11).

The pledget 40 may generally be a mesh-like, woven, or braided component that can assist with distributing pressure from the suture(s) 12, 14 along a greater surface area of the tissue T. The sutures 12, 14 are configured to extend through the respective pledget 40, and, during the tensioning process may be drawn through the pledget 40. The pledget 40 may be constructed of polytetrafluoroethylene (PTFE), such as Teflon®, or may be constructed of a fiber material, such as polyester.

Referring to FIGS. 3-7, the suture assembly 10 may include the pledget 40, illustrated as the flat pledget 40A, supporting at least one suture 12. The suture assembly 10 may include a single suture 12 forming the two preformed or predefined loops 16, 18, which may be utilized to attach the replacement valve V to the tissue T and press the implant clip 30 against the valve V. The suture 12 may include a first suture portion 50 including the first end 22 and forming the first predefined loop 16, as well as a second suture portion 52 including the second end 24 and the second predefined loop 18. The first end 22 and the second predefined loop 18 may be disposed adjacent to one another, while the second end 24 and the first predefined loop 16 may be disposed adjacent to one another. In this way, segments of the first suture portion 50 can be on opposing sides of the pledget 40, while segments of the second suture portion 52 can be on opposing sides of the pledget 40.

The suture 12 may be configured to extend through the pledget 40 multiple times to form the tail ends 22, 24 and the loops 16, 18 at opposing sides of the pledget 40. The first end 22 and the second predefined loop 18 may be spaced from the second end 24 and the first predefined loop 16 by the pledget 40. For example, as illustrated in FIG. 3, the first end 22 and the second loop 18 can be arranged at a left side of the pledget 40 while the second end 24 and the first loop 16 can be arranged at a right side of the pledget 40. Moreover, the ends 22, 24 and the loops 16, 18 may be arranged to extend from an abutting surface of the pledget 40, which may be configured to engage or abut the tissue T.

The first end 22 may be a first tail extending from the pledget 40. The suture 12 may extend from the first end 22, through the pledget 40, and then from the pledget 40 at an opposing side or end. The suture 12 may then form the first loop 16 and extend back through the pledget 40 toward the first end 22. The suture 12 may form the second loop 18 proximate to the first end 22, extend through the pledget 40, and then extend from the pledget 40 to form the second end 24 or second tail proximate to the first loop 16. Accordingly, the full extension of the suture 12 may be: from the first end 22, extending through the pledget 40, forming the first loop 16, extending through the pledget 40, forming the second loop 18, extending through the pledget 40, and to the second end 24.

Referring to FIGS. 3 and 4, the suture assembly 10 may also include at least one locking feature 36. In certain aspects, each predefined loop 16, 18 may be associated with a respective locking feature 36 (e.g., first and second locking features 36). Alternatively, each predefined loop 16, 18 for the suture 12 may be associated with the same locking feature 36. The locking feature 36 may be configured to retain one or both of the predefined loops 16, 18 in the tensioned state in response to the tensioning process, as described herein.

In certain aspects, the locking feature 36 may be configured as at least one spliced segment 56 in the suture 12. The spliced segment 56 may be formed from two limbs or otherwise interlaced or woven to allow the suture 12 to extend through itself at the spiced segment 56. The spliced segment 56 may be between about five millimeters and about six millimeters in length along a length of the suture 12. The suture 12 may include a single spliced segment 56, and the suture 12 may extend through the spliced segment 56 twice (e.g., for locking both loops 16, 18). Alternatively, each portion 50, 52 of the suture 12 may include a separate spliced segment 56 for more separately tensioning the loops 16, 18, respectively.

The suture 12 may have a greater thickness or width proximate to the spliced segment(s) 56 as part of or in conjunction with the locking feature 36. In the illustrated configuration, the suture 12 includes two thickened portions 58 for locking each of the loops 16, 18 in the tensioned state. However, it is contemplated that a single thickened portion 58 may be utilized without departing from the teachings herein. The locking feature 36, including the spliced segment(s) 56 and the thickened portion(s) 58, may be formed by the suture 12 in sections of the suture 12 extending through the pledget 40.

The first thickened portion 58 may be arranged at a connection between the first end 22 and the first loop 16, and the second thickened portion 58 may be arranged at a connection between the second end 24 and the second loop 18. The thickened portions 58 may include padding or stuffing, which may be additional suture material stuffed within the suture 12 to increase the width or thickness to assist with locking the suture 12 in the tensioned state. In response to the suture 12 being drawn through the spliced segment(s) 56 during the tensioning process, as described herein, the thickened portions 58 of the suture 12 can bunch or catch to create a locking trap. This locking trap and the resistance caused by the thicker stuffed portion(s) 58 of the suture 12 may lock the suture 12 in the tensioned state with the two loops 16, 18 tensioned over the implant clip 30 (see FIG. 4). Other configurations of locking features 36, such as the one described with respect to FIG. 14, may be utilized with the suture 12 without departing from the teachings herein.

Referring now to FIGS. 4-7, the suture assembly 10 may include the two needles 26, 28 for carrying the suture 12 through the tissue T and the valve V at two locations. The two locations may be determined by a size of the pledget 40 and/or a size of the implant clip 30. In various aspects, the suture assembly 10 can include two connecting sutures 64 for coupling the loops 16, 18 with the needles 26, 28, respectively. The connecting sutures 64 may be threaded around or form a loop through a respective one of the predefined loops 16, 18. The connecting sutures 64 can have both ends or tails coupled to the respective needle 26, 28, consequently coupling the predefined loops 16, 18 with the needles 26, 28. The connecting sutures 64 may be constructed similarly to the suture 12. Generally, the connecting sutures 64 may loop around a midsection of the predefined loops 16, 18 for the loops 16, 18 to have a similar length as the ends 22, 24 for more efficiently passing the suture 12 through the tissue T and the valve V.

The first end 22 and the second predefined loop 18 may be coupled with the first needle 26, while the second end 24 and the first predefined loop 16 may be coupled with the second needle 28. Accordingly, both ends 22, 24 and both predefined loops 16, 18 may be coupled with the needles 26, 28, respectively, for being carried through the tissue T and the valve V. The use of two needles 26, 28 can allow the suture assembly 10 to be packaged in a preassembled configuration with the preformed loops 16, 18, ready for application in a sterile setting. Further, the use of the two needles 26, 28 may allow the preformed loops 16, 18 to be passed through the tissue T and the valve V, which may provide a more efficient process for using the suture assembly 10 than shuttling processes for forming loops or stitches.

The suture assembly 10 may also include the implant clip 30, which may be arranged over/on the valve V for pressing the valve V against the tissue T. In certain aspects, the implant clip 30 may be arranged to abut an outer or perimeter ring of the valve V. The implant clip 30 may be pressed into the replacement valve V by the suture 12, which, consequently, presses the replacement valve V against the annulus A to better seal the replacement valve V to the tissue T. The implant clip 30 may assist with more evenly distributing the compressing/sealing force along the outer side of the valve V to provide a more even and tighter seal between the valve V and the tissue T. Additionally, the implant clip 30 may distribute the force caused by the tensioned suture(s) 12 over a greater area, similar to the pledget 40. Accordingly, the suture assembly 10 may have features on each side of the tissue T-valve V engagement that distribute pressure, which can also assist in forming a better, more even seal.

The implant clip 30, also referred to as a clip, a clamp, a connector, or an implant, may have a variety of configurations for engaging one or multiple loops 16, 18 and pressing against the valve V. In various aspects, the implant clip 30 may be constructed of polyetheretherketone (PEEK) or another similar material. The implant clip 30 may include or be constructed of polytetrafluoroethylene (PTFE) and/or polyester. The implant clip 30 may be configured to at least temporarily change shape, such as by being elastically deformable, to load the suture loops 16, 18, and then return to an initial or original state/shape to retain the engagement with the suture loops 16, 18.

As illustrated in FIGS. 5-7, the implant clip 30 can be a slotted ring 30A. The ring 30A may generally be circular or oblong but may have other shapes without departing from the teachings herein. The slotted ring 30A may define an insertion slot 70 with outer beveled edges 72. The beveled edges 72 can widen the insertion slot 70 at an outer edge of the ring 30A to assist with loading or inserting the loops 16, 18 into the slotted ring 30A. In various aspects, the slotted ring 30A may be configured to deform or elastically deform (e.g., twist or widen) to widen the insertion slot 70 for receiving or loading the loops 16, 18 on the slotted ring 30A. The slotted ring 30A may then again be deformed or return to its original shape to close or generally close the insertion slot 70 and retain the loaded loops 16, 18 on the slotted ring 30A. This may assist in retaining the engagement between the suture loops 16, 18 and the slotted ring 30A as the loops 16, 18 are being tensioned over the slotted ring 30A.

Each of the predefined loops 16, 18 may have a single limb that is inserted through the insertion slot 70, allowing each of the loops 16, 18 to extend over/around the slotted ring 30A. The slotted ring 30A may be arranged between the two predefined loops 16, 18 for generating opposing pulling forces on the slotted ring 30A with the loops 16, 18 during the tensioning process and in the tensioned state. The loops 16, 18 may each be configured to be tensioned over the slotted ring 30A to apply outward forces on the ring 30A and/or pressing (e.g., toward the pledget 40) forces on the ring 30A to press the ring 30A into the valve V and, consequently, the valve V into the tissue T. The outward/opposing forces on the ring 30A may assist in reducing or preventing movement of the ring 30A relative to the valve V in response to the loops 16, 18 being held under tension. The configuration of the implant clip 30 as the slotted ring 30A may minimize the size of the implant ring 30A and locations where the suture 12 can disengage from the ring 30A during the tensioning process.

As illustrated in FIG. 8, the implant clip 30 may be configured as a notched clip 30B with a “figure 8” or “dog bone” shape having opposing side notches 74, 76 at a central area of the notched clip 30B. In such configurations, the loops 16, 18 may be configured to be inserted or arranged in each opposing notch 74, 76, extending over the notched clip 30B between the two notches 74, 76. The notches 74, 76 may be configured to retain the engagements between the implant clip 30B and the loops 16, 18. The notches 74, 76 may remain slightly open or the clip 30B may deform (e.g., elastically deform) to open the notches 74, 76 for the insertion of the loops 16, 18 before returning to a generally closed initial state. Similar to the ring 30A (see FIG. 5), the loops 16, 18 may be configured to be tensioned over the implant clip 30B with the suture 12 extending through the notches 74, 76, applying the outward/opposing forces on the notched clip 30B and/or the pressing force to press the notched clip 30B toward or against the valve V. The notched clip 30B may allow for more efficient loading of the loops 16, 18 onto the implant clip 30B, and the ring 30A may retain a better engagement with the loops 16, 18.

The ring 30A and the notched clip 30B may each be configured to retain the suture 12 on the respective clip 30, reducing the disengagement of the suture 12 during the tensioning process. The slot 70 and the notches 74, 76 may be sufficient in size or the clips 30 may deform to allow the insertion of the loops 16, 18 into the clip 30 and may be sufficiently small to retain the suture 12 on the clip 30. Further, similar to the notches 74, 76, the slot 70 may be positioned between the loops 16, 18. In other words, the notches 74, 76 and the slot 70 may be arranged in a location spaced from the loops 16, 18 to reduce the likelihood that the suture 12 disengages from the clip 30. The loops 16, 18 may engage the rounded shape of the ring 30A on either side of the slot 70. Similarly, the loops 16, 18 may extend over a cross feature of the clip 30B and engage rounded ends coupled to the cross feature with ends defining the notches 74, 76. This arrangement may allow the suture 12 to extend over the clips 30 and provide tensioning, pulling, or opposing forces on the clips 30 during and after the tensioning process while remaining engaged with the clip 30. Closing the slot 70 and/or the notches 74, 76 can further reduce the chance of disengagement during the tensioning process.

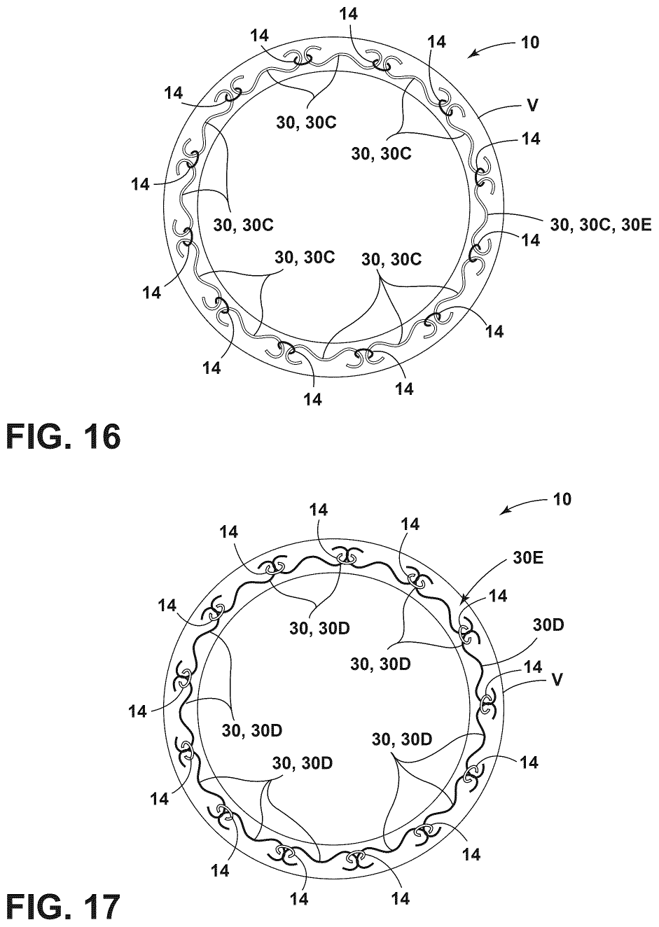

The implant clip 30 may abut a top or outer surface of the valve V, such as a valve ring, and press the valve V against the tissue T. The implant clip 30 may be advantageous for providing increased pressure or better distributing the pressure to form a better seal between the valve V and the tissue T. The implant clip 30 may apply the pressure over a greater area compared to a suture stitch to assist in providing a more consistent fluid-tight seal between the tissue T and the valve V around the annulus A. Other configurations of the implant clip 30 may be utilized, such as a C-shaped clip 30C (see FIG. 16) or an S-shaped clip 30D (see FIG. 17), without departing from the teachings herein.

Referring again to FIGS. 5-8, in operation, the suture assembly 10 may be arranged under the annulus A, extend through the tissue T and the valve V, and press the implant clip 30 against the top/outer side of the valve V for attaching the valve V to the tissue T and assisting with the seal therebetween. For the replacement valve V procedure, the pledget 40 may be arranged along an underside or inner surface of the tissue T or under the annulus A. The suture assembly 10 may include the two needles 26, 28 to pass the ends 22, 24 of the suture 12 and the predefined loops 16, 18 of the suture 12 through the tissue T from a first side to an opposing second side and through the replacement valve V. The first needle 26 can be inserted through the tissue T from the underside of the annulus A and then through the replacement valve V, which may bring the first end 22 of the suture 12 and the second predefined loop 18 through the tissue T and the replacement valve V. Similarly, the second needle 28 can be inserted through the tissue T from the underside of the annulus A and then through replacement valve V, which may bring the second end 24 and the first predefined loop 16 through the tissue T and the valve V.

The ends 22, 24 and the predefined loops 16, 18 extending through the tissue T may be spaced apart by approximately the length of the pledget 40 and/or the implant clip 30. In certain aspects, the spacing may be greater than the size of the implant clip 30 to provide an increased outward force or opposing forces on the implant clip 30, which can reduce movement of the implant clip 30. In other aspects, the spacing may align with the outer edges of the implant clip 30 to be able to position two implant clips 30 adjacent to one another without significantly affecting the adjacent sutures 12.

The two needles 26, 28 and the corresponding engagement to the suture 12 may allow the pledget 40 to remain under the annulus A to distribute pressure caused by the suture 12 in the tensioned state. Further, use of the two needles 26, 28 can assist with strengthening the engagement between the tissue T and the valve V to provide a better seal and compression between the replacement valve V and the tissue T. For example, with an aortic valve replacement, there is generally a high gradient of blood flow that moves through the aortic valve V into the body of the patient to be able to pump blood through the extremities of the patient. The two pass-throughs of the suture assembly 10 through the tissue T and replacement valve V may assist in increasing the fluid-tight seal between the tissue T and replacement valve V. After the suture 12 has been passed through the tissue T and the valve V, the needles 26, 28 and the connecting sutures 64 may be removed from the suture 12.

The implant clip 30 may be arranged over the valve V with the loops 16, 18 loaded onto the clip 30. The tensioning or pulling force may be applied to both ends 22, 24 of the suture 12. In response to the pulling force, the suture 12 can be pulled through the pledget 40 with the predefined loops 16, 18 decreasing in size in response to the tensioning process. The pulling force may continue to be applied until there is minimal or no slack remaining in the suture 12 with the predefined loops 16, 18 tightened or tensioned over the implant clip 30. As the loops 16, 18 are tensioned over the implant clip 30, the first portion 50 and the second portion 52 of the suture 12 may be drawn through the spliced segment(s) 56, causing the stuffing of the thickened portion(s) 58 to bunch/catch and lock the suture 12 in the tensioned state. This locking feature 36 and the resistance caused by the thicker stuffed portion 58 of the suture 12 may retain the suture 12 in the tensioned state with the two loops 16, 18 tensioned over the implant clip 30. The suture 12 may be re-tensioned, with the locking feature 36 retaining the predefined loops 16, 18 in the newly tensioned state after each re-tensioning.

Referring to FIGS. 7 and 8, as previously noted, the pledget 40 may have different configurations, including the flat pledget 40A. The flat pledget 40A has a generally rectangular or square profile and may have a thin, generally flat body. In such configurations, the pledget 40A may be formed of a woven or rigid structure through which the suture 12 can extend and travel. The flat pledget 40A may have a thinner profile for minimizing the size of the suture assembly 10.

In the configuration illustrated in FIGS. 9 and 10, the pledget 40B is exemplified as a braided or interlaced, tubular structure. The braided structure may be folded to form two layers 90, 92 with openings into the tubular structure oriented in the same direction (illustrated as a right direction). Accordingly, the folded pledget 40B may form a first layer 90 that can engage the underside of the tissue T and a second layer 92 that can engage the first layer 90. The suture 12 may be configured to extend through both layers 90, 92 of the pledget 40B to press the pledget 40B against the tissue T. The suture 12 may extend entirely through both layers 90, 92. Alternatively, the suture 12 may extend partially through the second layer 92. In such examples, the suture 12 may extend along the second layer 92, such as through a lumen or a wall defining the lumen.

In various aspects, both portions 50, 52 of the suture 12 may extend across the second layer 92. Alternatively, the first portion 50 may extend across the first layer 90 and the second portion 52 may extend across the second layer 92, which may separate the spliced segment(s) 56 and/or the locking features 36 for the two predefined loops 16, 18. The folded pledget 40B may provide additional density and depth for supporting or forming the locking feature 36 and the movement of the suture 12.

As illustrated in FIG. 11, the suture assembly 10 may include the pledget 40C with a ring or annular shape that generally follows the shape of the annulus A and/or the valve ring of the valve V. The annular pledget 40C may form a single structure that can be positioned below the annulus A and support multiple sutures 12. Accordingly, a single annular pledget 40 may be used with many, most, or all the sutures 12 to be utilized for the valve replacement procedure. For example, the annular pledget 40C may support between 12 and 17 sutures 12 resulting in between 24 and 34 predefined loops 16, 18 and needles 26, 28. This configuration may be advantageous for maximizing efficiency when utilizing multiple sutures 12 during the valve replacement procedure. The sutures 12 may be preset for select locations on the annulus A. Additionally, this configuration may be advantageous for further distributing pressure along the annulus A from the tensioned sutures 12 and the implant clips 30. The pledget 40 may also form different shapes and/or lengths that support different numbers of sutures 12 without departing from the teachings herein.

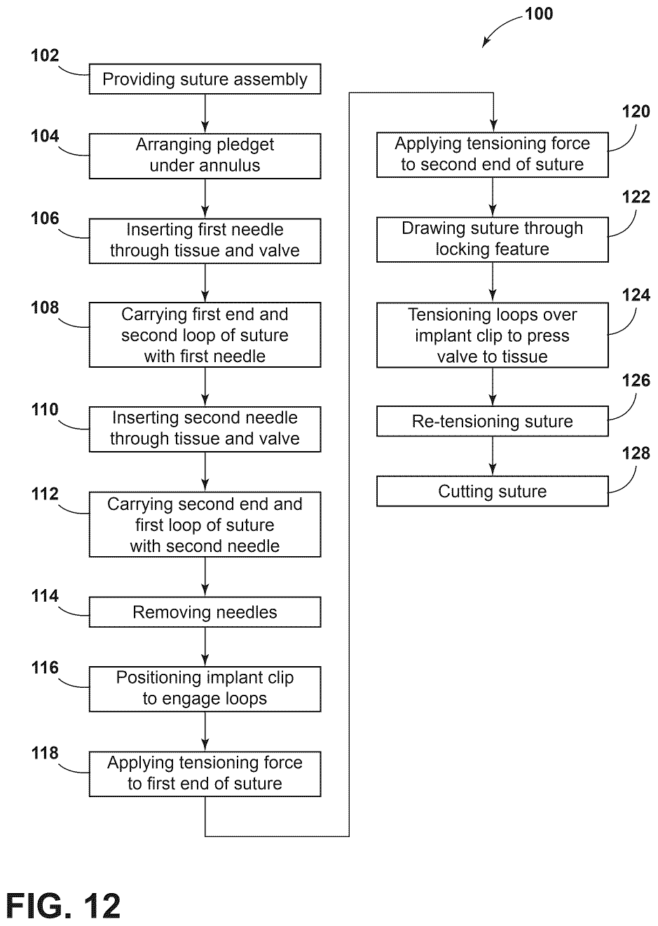

Referring to FIG. 12, as well as FIGS. 1-11, a method 100 of using the suture assembly 10 for the valve V replacement procedure includes providing the suture assembly 10 that includes the pledget 40 and at least one suture 12 coupled with the two needles 26, 28 (102). In the example shown, the suture 12 can be pre-loaded into the pledget 40 to have two ends 22, 24 and two preformed, tensionable loops 16, 18. As previously described, the leaflets of the aortic valve may be removed from the patient, and the replacement valve V may be provided. The pledget 40 can be arranged on the underside of the annulus A where the replacement valve V is to be attached (104). The first needle 26 can be inserted through the tissue T and the replacement valve V (106). The first needle 26 may be configured to carry the first end 22 and the second predefined loop 18 through the tissue T and the replacement valve V (108).

Similarly, the second needle 28 may be inserted through the tissue T and the replacement valve V at a location spaced from the location where the first needle 26 is inserted (110). The second needle 28 may be configured to carry the second end 24 and the first predefined loop 16 through the tissue T and the replacement valve V (112). At this stage, the ends 22, 24 and the predefined loops 16, 18 of the suture 12 may be on an opposing side of the tissue T and the valve V compared to the pledget 40. Tension may be applied by pulling both needles 26, 28 to pull the suture 12 fully through the tissue T and the valve V, as well as move the pledget 40 to engage the tissue T.

The ends 22, 24 of the sutures 14 and the connecting sutures 64 may be cut to remove the needles 26, 28 from the suture 12 (114). In certain aspects, the suture 12 may have indicators proximate to the ends 22, 24 to identify a cutting location or area. In removing the needles 26, 28, the ends 22, 24 may be separated from the adjacent predefined loops 16, 18. The implant clip 30 may be provided and positioned over the valve V between the loops 16, 18 (116). The loops 16, 18 may be hooked over, looped through, or otherwise engaged with the implant clip 30. For example, with the slotted ring 30A, a single limb from each loop 16, 18 may be inserted through the insertion slot 70 to extend around the ring 30A. With the notched clip 30B, the single limb from each loop 16, 18 may be arranged in each notch 74, 76 of the implant clip 30 with the loops 16, 18 extend over a body (e.g., a cross feature) of the implant clip 30B between the notches 74, 76. It is also contemplated that the two loops 16, 18 may engage separate implant clips 30 similar to the single loops 20 described with respect to FIGS. 13-17.

The pulling or tensioning force may be applied to the first end 22 of the suture 12 (118). The tensioning force may increase the length of the suture 12 proximate to the first end 22, while decreasing the size of the first predefined loop 16. This can adjust the configuration of the first suture portion 50. Similarly, the tensioning force may be applied to the second end 24 of the suture 12, pulling the second portion 52 of the suture 12 through the pledget 40 to decrease the size of the second predefined loop 18 (120).

The suture 12 may be drawn through the locking feature 36 (122). The tensioning force may draw the sections of the suture 12 that define the preformed loops 16, 18 into/through the pledget 40 and through the locking feature 36. In certain aspects, the sections of the suture 12 forming the loops 16, 18 may be drawn through the spliced segment(s) 56, and the thickened portion(s) 58 of the suture 12 proximate the spliced segment(s) 56 may bunch to form the locking trap. The locking feature 36 may be configured to automatically lock the suture 12 in the tensioned state. Further, the locking feature 36 may be configured to reduce or prevent loosening of the suture 12 (e.g., movement in a direction opposing the tensioning force). Each of the predefined loops 16, 18 may be configured to be retained in the tensioned state by the locking feature 36.

The tensioning force can continue to be applied to tension the loops 16, 18 over the implant clip 30, pressing the implant clip 30 into the valve V and the valve V into the tissue T to form the seal (124). Additional suture assemblies 10 may follow this process. For example, steps 102-124 may be repeated for using multiple sutures 12 with separate pledgets 40. Steps 106-124 may be repeated for multiple sutures 12 using a single pledget 40, such as the annular pledget 40C. The number of sutures 12 or suture assemblies 10 may depend on the patient and/or the procedure for securely attaching the placement valve V to the tissue T.

One or more of the sutures 12 may be re-tensioned (126). The locking features 36 may automatically lock the sutures 12 with each re-tensioning. Re-tensioning may provide for better placement of the valve V relative to the tissue T, which may result in a better seal therebetween. A pusher/cutter device may be used to apply pressure to the implant clip 30 or the valve V while tensioning and re-tensioning the sutures 12 (128). This may be advantageous for reducing displacement of the valve V and reducing slack from the suture assemblies 10.

The sutures 12 may be cut at each end 22, 24 to be flush with the valve V. The use of the pusher/cutter device may provide a better seal between the tissue T and the replacement valve V, as well as more fully tension the sutures 12 to then be cut flush with the replacement valve V. The locking features 36 can retain the loops 16, 18 of the suture 12 in the tensioned state, forming stitch configurations over the implant clip 30 to provide the pressing force on the valve V. The method 100 may be modified or conducted in any order, with steps omitted, repeated, performed concurrently, etc. without departing from the teachings herein.

The dual loop suture 12 may have two preformed loops 16, 18 for engaging one or more implant clips 30, which can increase efficiency during the surgical procedure. In this regard, the dual loop suture 12 may be prepackaged with the loops 16, 18 for forming the stich over the implant clip 30. The shuttling process for forming stitches may be omitted or skipped due to the preformed loops 16, 18, which can increase efficiency for the surgical team and reduce time for the procedure.

With reference to FIGS. 13-16, the suture assembly 10B may include at least one single-loop suture 14 having the preformed or predefined loop 20. Typically, the suture assembly 10B may include multiple separate sutures 14, which may be coupled to individual pledgets 40 or a single pledget 40. The multiple sutures 14 may be arranged around the annulus A (see FIG. 1) with loops 20 from adjacent sutures 14 engaging an interconnected implant clip 30. The single loop suture 14 may increase flexibility in the arrangement of the sutures 14 and engagement of the clips 30. Additionally, the single loop sutures 14 may increase manufacturing efficiency by forming a single loop 20, rather than forming the two loops 16, 18 as described with respect to FIGS. 3-11.

Referring to FIGS. 13-15, each suture 14 generally includes a single predefined loop 20, which may be arranged adjacent to one another for engaging the same clip 30 or multiple adjacent clips 30. Stated differently, with multiple sutures 14, the first predefined loop 20 for engaging the implant clip 30 may be formed by a first suture 14, and the second predefined loop 20 for engaging the implant clip 30 may be formed by a second suture 14. Each suture 14 may include the first end 22 coupled or fixed to the pledget 40 via a base knot 152 (e.g., a fixed end 22). The suture 14 may extend out of the pledget 40 to form the loop 20, back through the pledget 40, and again out of the pledget 40 to the second end 24 (e.g., a tail end 24). In this way, the tail end 24 may be spaced from the loop 20 by the pledget 40. Each of the second end 24 and the predefined loop 20 may extend from the abutting surface of the pledget 40 to be inserted through the tissue T and the valve V. The single loop suture 14 may be utilized with the flat pledget 40A, the folded pledget 40B, and/or the annular pledget 40C for increased customizability of the suture assembly 10.

The suture 14 may be coupled with at least one needle 26, though the suture 14 may typically be coupled with two needles 26, 28 for carrying the suture 14 through the tissue T and the valve V. In examples with the two needles 26, 28, the first needle 26 may be coupled to the tail end 24 and the loop 20 may be coupled to the second needle 28 via the connecting suture 64. This may allow the tail end 24 and the predefined loop 16 to be inserted through the tissue T and the valve V at different locations, which may assist in distributing the pressure formed from the tensioned suture 14. It is contemplated that the tail end 24 and the predefined loop 16 may be coupled with a single needle 26 without departing from the teachings herein.

Each suture 14 may include or extend through a respective locking feature 36 for retaining the suture 14 in the tensioned state in response to the tensioning process. As illustrated in FIG. 13, the locking features 36 may be configured as the spliced segment 56, with the suture 14 being woven, interlaced, or otherwise extending through itself. In such examples, the suture 14 may also include the thickened portion 58 proximate to the spliced segment 56 for bunching during the tensioning process and forming the locking trap as described herein. In this way, the suture 14 can extend through the spliced segment 56, and the section of the suture 14 that forms the loop 16 can be pulled through the spliced segment 56 during the tensioning process. The spliced segment 56 may reduce the profile of the suture assembly 10 and the number of components in the suture assembly 10 while retaining the suture 14 in the tensioned state.

As illustrated in FIG. 14, in certain aspects, the locking feature 36 may be configured as a grommet 170. Each suture 14 may be associated with a separate grommet 170 or several sutures 14 may be associated with a single grommet 170. The grommet 170 may be disposed in and coupled with the pledget 40. The grommet 170 may include inner projections or barbs 172 that are angled to allow the respective suture 14 to be passed in one direction and not the opposing direction. In this way, the inner barbs 172 may allow movement through the grommet 170 in a single direction to tension the suture 14, while reducing or preventing movement that loosens the suture 14 (e.g., in the opposing direction).

The grommet 170 may include outer projections or barbs 174 that engage the braided or woven structure of the pledget 40. This engagement may retain the grommet 170 in the predefined location relative to the pledget 40, including as tensioning force is applied to the suture 14 being pulled through the grommet 170. The fixed location of the grommet 170 may generally assist with more fully tensioning the suture 14 and locking the suture 14 under tension over the implant clip 30. The grommet 170 may increase manufacturing efficiency for the suture 14 by omitting the process for forming the spliced segment 56. The grommet 170 may be utilized with any configuration of the pledget 40 without departing from the teachings herein.

Referring now to FIGS. 15-17, two sutures 14 may engage a single implant clip 30, similar to the two loops 16, 18 of the single suture 12 described with respect to FIGS. 3-8. The two sutures 14 may allow for more precise spacing between adjacent loops 20 based on the implant clip 30. For example, implant clips 30 may have different sizes or lengths, and the sutures 14 can be arranged to engage each end of the clip 30. This may provide for more customizable implants 30.

Further, each loop 20 may engage two adjacent clips 30. This may allow a single loop 20 to be tensioned over adjacent clips 30 for pressing the adjacent clips 30 into the valve V. This configuration may reduce the number of sutures 14 used for the procedure. Additionally, the single loop sutures 14 may be utilized for forming an interconnected implant ring 30E. The implant ring 30E may be formed of multiple implant clips 30 arranged adjacent to one another along the perimeter of the valve V. The implant clips 30 may be arranged adjacent to one another and coupled together via sutures 14 or may be directly coupled together (e.g., at a joint). The implant ring 30E may be rigid and/or may have a select degree of flexibility for conforming to the shape of the valve ring.

The implant clips 30 can be coupled together via the sutures 14 to form the interconnected and flexible implant ring 30E. The implant clips 30 forming the implant ring 30E may be the C-shaped implant clips 30C, as illustrated in FIG. 16, or the S-shaped implant clips 30D, as illustrated in FIG. 17. The implant clips 30 that form the implant ring 30E may have two curved or substantially enclosed ends for engaging the two adjacent sutures 14. The implant clips 30 may also be the ring 30A illustrated in FIG. 5, the clip 30B illustrated in FIG. 8, another practicable configuration. Other shapes or configurations of the implant clips 30 may have two insertion slots 70 (see FIG. 5), notches 74, 76 (see FIG. 8), two hook-like features, an oblong or overall shape, a figure “8” or lemniscate shape, a “dog bone” shape, etc. The curved or enclosed ends of the clips 30 may be utilized for retaining the engagement with the loops 20 during and after the tensioning process. The enclosed ends may be advantageous for retaining the sutures 14 on the clips 30 during the tensioning process.

Generally, each suture 14 may be configured to engage two adjacent implant clips 30 to couple the implant clips 30 together as well as to the valve V and tissue T. For example, as illustrated in FIG. 15, the tail end 24 and the predefined loop 20 of the first suture 14 can be inserted through the tissue T and the valve V. The tail end 24 and the predefined loop 20 of the second suture 14 can be inserted through the tissue T and the valve V spaced from the first suture 14. Two implant clips 30 may be provided and arranged over the valve V on opposing sides of the first suture 14 (e.g., illustrated as right and left sides of the left suture 14), and one of the two implant clips 30 may be arranged between the first suture 14 and the second suture 14. An additional or third implant clip 30 may be provided on the opposing side of the second suture 14 (e.g., illustrated as a right side of the right suture 14). This forms an alternating pattern of: first implant clip 30, first suture 14, second implant clip 30, second suture 14, third implant clip 30, etc. This repeated pattern may be formed around the valve V to form the implant ring 30E. It is also contemplated that this repeated pattern may be used to form a chain of implant clips 30, with the end sutures 14 engaging the single distal clips 30, respectively.

The first suture 14 may be coupled to a first side of the first implant clip 30 and a second side of the second implant clip 30. The second suture 14 may be coupled to a first side of the second implant clip 30, opposite the first suture 14, and a second side of the third implant clip 30. Accordingly, the first and second sutures 14 may each engage two adjacent implant clips 30. Further, the first and second sutures 14 may engage opposing sides of the implant clip 30 disposed therebetween. Each implant clip 30 may be arranged between two adjacent sutures 14. This configuration may form the interconnected implant ring 30E, which may be advantageous for providing more consistent or even pressure around the valve V. In various aspects, the interconnected suture 12, 14 and implant clip 30 system may utilize any configuration of the implant clips 30 or sutures 12, 14 described herein.

Each of the sutures 14 may be configured to be tensioned over the two adjacent implant clips 30. The tensioning may be performed in sequence, in a pattern, etc. until each of the sutures 14 is tensioned to press the implant ring 30E into the valve V. The implant ring 30E may distribute the pressure from the sutures 14 around the annulus A. When the annular pledget 40C is used with the implant ring 30E, the suture assembly 10 may more evenly distribute the pressure on both the underside of the annulus A and over the valve V. The pressure distribution may provide a better fluid-tight seal between the tissue T and the valve V.

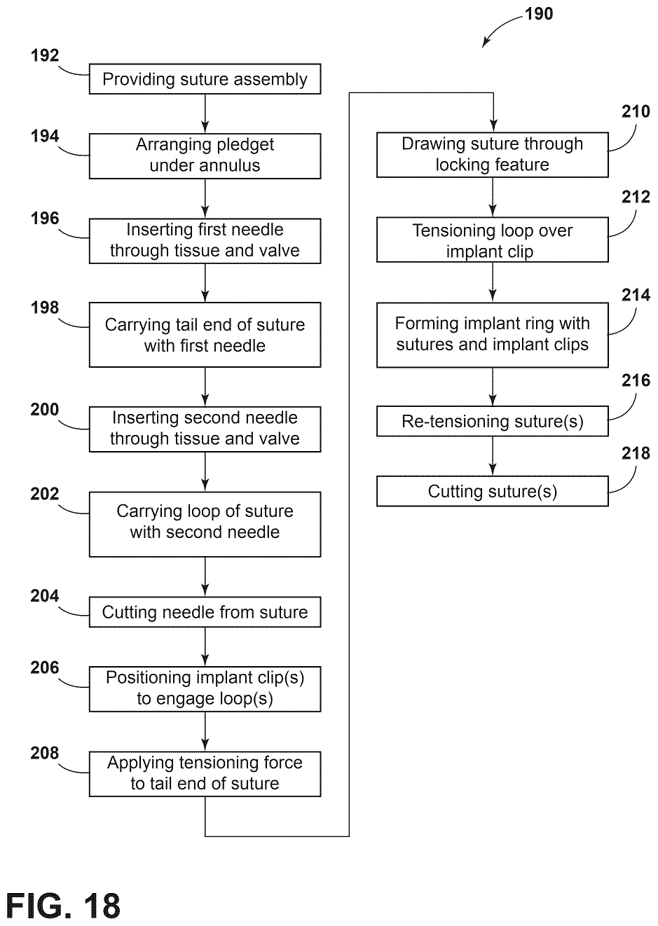

Referring to FIG. 18, as well as FIGS. 1-17, a method 190 of using the suture assembly 10 with the single loop suture 14 for the valve replacement procedure includes providing the suture assembly 10 or suture assemblies 10 that includes the pledget(s) 40 and multiple single loop sutures 14 (192). Generally, each suture 14 may be coupled with two needles 26, 28. Each suture 14 can also be pre-loaded into the pledget 40 to have the preformed, tensionable loop 20. As previously described, the leaflets of the aortic valve V may be removed from the patient, and the replacement valve V may be provided. The pledget 40 can be arranged on the underside of the annulus A where the replacement valve V is to be attached (194). The first needle 26 may be inserted through the tissue T and the replacement valve V (196). The first needle 26 may be configured to carry the tail end 24 of the suture 14 through the tissue T and the replacement valve V (198).

Similarly, the second needle 28 may be inserted through the tissue T and the replacement valve V (200). The second needle 28 may be configured to carry the predefined loop 20 of the suture 14 through the tissue T and the replacement valve V (202). Steps 196-202 may be repeated for the second suture 14 (e.g., using an additional two needles 26, 28) and any subsequent sutures 14. At this stage, the ends 24 of the sutures 14 and the predefined loops 20 may be on an opposing side of the tissue T and the valve V compared to the pledget(s) 40. Tension may be applied by pulling the needles 26, 28 to pull the sutures 14 fully through the tissue T and the valve V and move the pledget(s) 40 to engage the tissue T.

The tail ends 24 of the sutures 14 and the connecting sutures 64 may be cut to remove the needles 26, 28 from the sutures 14 (204). In certain aspects, the sutures 14 may have indicators proximate to the ends 24 to identify a cutting location or area. The implant clips 30 may be provided and positioned over the valve V adjacent to the loops 20 (206). In certain aspects, the loops 20 of two adjacent sutures 14 may engage a single implant clip 30. In such examples, the loops 20 of the adjacent sutures 14 may engage the common implant clip 30 on opposing sides (e.g., forming the first and second loops 20 to engage the common clip 30). Additionally or alternatively, the loop 20 of a single suture 14 may engage adjacent implant clips 30. The loops 20 of sutures 14 may be hooked over, looped through, or otherwise engage with the respective implant clip(s) 30.

The pulling or tensioning force may be applied to the tail end 24 of the first suture 14 (208). The tensioning force may increase the length of the tail end 24 of the first suture 14 while decreasing the size of the first predefined loop 20. Similarly, the tensioning force may be applied to the tail end 24 of the additional sutures 14, pulling the sutures 14 through the pledget(s) 40 to decrease the size of the predefined loops 20.

The sutures 14 may be drawn through the respective locking features 36 (210). The tensioning force may draw the sections of the sutures 14 that define the preformed loops 20 into/through the pledget 40 and through the locking features 36. In certain aspects, the sutures 14 may be drawn through the spliced segment 56, and the thickened portions 58 of the sutures 14 proximate to the spliced segments 56 may bunch to form the locking trap. In other aspects, the sutures 14 may be drawn through the grommets 170 to engage the inner barbs 172. The locking features 36 may be configured to automatically lock the sutures 14 in the tensioned state. Further, the locking feature 36 may be configured to reduce or prevent loosening of the suture 14 (e.g., movement in a direction opposing the tensioning force). Each of the predefined loops 20 may be configured to be retained in the tensioned state by the locking features 36.

The tensioning force can continue to be applied to tension the loops 20 over the implant clips 30, pressing the implant clips 30 into the valve V and the valve V into the tissue T (212). Additional suture assemblies 10 may follow this process. For example, steps 194-212 may be repeated for using multiple sutures 14 with separate pledgets 40. Steps 196-212 may be repeated for multiple sutures 14 using a single pledget 40, such as the annular pledget 40C. The number of sutures 14 or suture assemblies 10 may depend on the patient and/or the procedure for securely attaching the placement valve V to the tissue T. The sutures 14 and implant clips 30 may continue to be added and coupled together to form the implant ring 30E or other chains of clips 30 (214). The implant ring 30E may abut the valve ring to apply pressure around the annulus A/valve V. The suture assembly 10 may include any practicable number of sutures 14 and implant clips 30 to form a sufficient seal between the valve V and the tissue T, including a sufficient number to form a full ring 30E around the replacement valve V.

One or more of the sutures 14 may be re-tensioned (216). The locking features 36 may automatically lock the sutures 14 with each re-tensioning. Re-tensioning may provide for better placement of the valve V relative to the tissue T, which may result in a better seal therebetween. The pusher/cutter device may be used to apply pressure to the implant clip 30 or the valve V while tensioning and re-tensioning the sutures 14 (218). This may be advantageous for reducing displacement of the valve V and reducing slack from the sutures 14.

The sutures 14 may be cut at each tail end 24 to be flush with the valve V. The use of the pusher/cutter device may provide a better seal between the tissue T and the replacement valve V as well as more fully tension the sutures 14 to then be cut flush with the replacement valve V. The locking features 36 can retain the loops 20 of the sutures 14 in the tensioned state, forming stitch configurations over the implant clips 30 to provide the pressing force on the valve V. The method 190 may be modified or conducted in any order, with steps omitted, repeated, performed concurrently, etc. without departing from the teachings herein.

The single loop sutures 14 may increase efficiency by allowing the surgical team to skip the shuttling process during the surgical procedure. The single loop 20 may also provide increased flexibility in the suture assembly 10, such as by utilizing spaced-apart implant clips 30 or by interconnecting the implant clips 30 with the sutures 14. The single suture 14 may also be manufactured more efficiently than the dual loop suture 12 by preforming a single loop 20 rather than two loops 16, 18.

Referring again to FIGS. 2-11 and 13-17, the suture assemblies 10 using the dual loop suture 12 and the single loop sutures 14 may be re-tensioned, which can allow for more accurate alignment between the valve V in the tissue T. Conventional sutures are generally tensioned a single time without the ability to be re-tension the suture, which can result in limitations in adjusting the valve V and, consequently, improper alignment between the valve V and the tissue T. Often during the valve replacement procedure, the sutures are initially tightened in a sequence around the valve V and, once tightened, the sutures can be knotted or a crimp can be crimped to lock or hold the sutures under tension. The sutures are generally fixed and may not be re-tensioned or adjusted. If the valve V shifted during the procedure, which can be common based on tension being applied in sequence, the valve V may be fixed in a slightly displaced position.

The re-tensioning that can be accomplished via the suture assembly 10 disclosed herein may provide for better alignment of the replacement valve V. For example, using the suture assemblies 10, the sutures 12, 14 may initially be tensioned over the implant clips 30 to attach the replacement valve V to the tissue T. During this process, the replacement valve V may become displaced due to the temporarily uneven tension of the sutures 12, 14, which can be more common and mechanical valves V. The sutures 12, 14 can then be re-tensioned multiple times during the valve V placement procedure to assist with balancing the repair or fixation of the replacement valve V and positioning the replacement valve V. Accordingly, the ability to re-tension the sutures 12, 14 may allow for better alignment, which can also result in a better fluid seal between the valve V and the tissue T.

The use of the implant clips 30 may also provide a more even seal between the valve V and the tissue T around the annulus A. For example, the implant clips 30 may generally engage an increased surface area of the valve V compared to a stitch formed by a suture 12, 14. The increased area distributes pressure and provides more even distribution of the force applied to the valve V and, consequently, the tissue T from the tensioned suture 12, 14. Additionally, fewer sutures 12, 14 may be utilized to provide the fluid tight seal by using the implant clips 30 to distribute the pressure. Further, in the interlocking or ring configurations 30E, the suture assembly 10 may provide a generally consistent compression along a longer portion of or around the entire perimeter of the valve V. The sutures 12, 14 may also provide increased flexibility through use of the combination of dual loop and single loop sutures 12, 14.

In certain aspects, the suture assembly 10 may also include smaller/thinner sutures 12, 14, which may reduce the footprint of the suture assembly 10. The sutures 12, 14 may be 0, 2-0, 4-0, or 5-0 sutures depending on the configuration of the suture assembly 10. The sutures 12, 14 may balance a thinness for carrying multiple suture 12, 14 limbs through the tissue T and valve V concurrently without having to contemplate a thickness for the shuttling process. The sizing of the sutures 12, 14 may provide a balance between being sufficiently narrow for the sutures 12, 14 to be carried or threaded through various features and having sufficient thickness to seal the valve V to the tissue T. The sutures 12, 14 may have a variety of configurations, such as tape-like, flat, tubular, tubular that compresses flat, etc. to balance between size and function. Portions of the sutures 14 may have different sizes, such as tapered ends 22, 24, which may occur through the manufacturing process. For example, the sutures 12, 14 may be formed of strands woven or interlaced together and fewer strands of material may be used to form the thinner sections.

Additionally, different portions of the sutures 12, 14 may have different configurations. For example, the sutures 12, 24 may have a tape portion that may become pledget-like, which can include the spliced segment(s) 56. The pledget-like section of the suture 12, 14 may be coupled with the pledget 40 and/or replace the pledget 40. The pledget 40 may have a variety of configurations including the flat, folded, and/or annular configurations. For example, the pledget 40 may include a braided jacket to be built into the round suture 12, 14 similar to a fiber tape. This may further reduce the size of the overall assembly 10 and the number of components utilized in the replacement procedure.

The sutures 12, 14 may have increased strength compared to conventional sutures. The sutures 12, 14 may be constructed of polyethylene. The polyethylene construction may increase the strength profile of the sutures 12, 14 compared to more conventional polyester sutures. The increased strength may be advantageous for applying the tensioning force to the sutures 12, 14.

As described herein, the first and second needles 26, 28 may be utilized to carry portions of the sutures 12, 14 through the tissue T and the valve V before being removed from the suture assembly 10. In some cases, different devices may be utilized in lieu of or in combination with the needles 26, 28 for carrying the sutures 12, 14 through the tissue T and the valve V without departing from the teachings herein. In such examples, one or more carrier or guide devices may function similar to the needles 26, 28 for engaging the sutures 12, 14, carrying the sutures 12, 14 or portions thereof, and/or disengaging from the sutures 12, 14 (e.g., for the tensioning process). Accordingly, the needles 26, 28 may be selectively implemented with other optional carrier, guide, or attachment devices to maneuver the sutures 12, 14.

For example, a suture passer device may be utilized with the suture assembly 10. The suture passer device may include a distal jaw with at least one movable jaw member and a movable needle. The jaw may be utilized for holding and moving the tissue T. The jaw may also support the needle, which can be selectively engaged with the suture 12, 14 to be passed. In certain aspects, a user may load one or both of the sutures 12, 14 or portions thereof (e.g., the ends 22 24 and associated loop 16, 18, 20) onto the needle of the suture passer. Additionally or alternatively, the suture passer may be configured to automatically re-load one or both of the sutures 12, 14 or portions thereof onto the needle. The suture passer may be used for carrying the sutures 12, 14 through the tissue T and the valve V without using an attached needle. In other words, the needle can remain as part of the suture passer, omitting the process of removing the needles 26, 28 from the suture assembly 10.

In an additional non-limiting example, an implant device can be used with the suture assembly 10 for placing the suture assembly 10 in the tissue T. The implant device may carry or house one or both sutures 12, 14 and/or the pledget 40. The suture assembly 10 may be housed in a sheath of the implant device. A needle tip of the implant device can be inserted through the tissue T and/or valve V and the user can deploy a portion of the suture assembly 10, such one section as the sutures 12, 14. This first section of the sutures 12, 14 may be the portions coupled with the first needle 26 in configurations utilizing the attached needles 26, 28. The user can then adjust the implant device to load a second section of the suture assembly 10 to be implanted. The second section of the sutures 12, 14 may be the portions coupled with the second needle 28 in configurations utilizing the attached needles 26, 28. The implant device can be removed from the tissue T and then re-inserted through the tissue T at a second location to deploy or carry the second section of the sutures 12, 14 through the tissue T at the second location to form the stitch arrangement.

The suture assembly 10 disclosed herein generally includes the attached needles 26, 28 for carrying the sutures 12, 14 through the tissue T and the valve V for forming the knotless stitch configuration. The suture assembly 10 may also utilize a device for passing the sutures 12, 14, such as the suture passer device or implant device, rather than the attached needles 26, 28. The suture assembly 10 may be configured substantially similarly with and without the attached needles 26, 28 for forming the knotless stitch configuration through tensioning the preformed loops 16, 18, 20, which can be tensioned and re-tensioned.

Referring to FIGS. 1-18, the suture assemblies 10 disclosed herein and use of the assemblies 10 may provide for a variety of advantages. For example, the suture assemblies 10 may include two needles 26, 28, which can allow the pledget 40 to be disposed under the annulus A. Also, the two needles 26, 28 may allow both of the sutures 12, 14 or different portions of the same suture 12, 14 to be directed through the tissue T and the replacement valve V, which can increase engagement and compression between the tissue T and the replacement valve V to provide a better fluid-tight or blood-tight seal for high-pressure blood flow. The pledget 40 may also provide for pressure distribution and reinforcement for the tissue T during the tensioning and re-tensioning processes. The implant clips 30 may also provide for pressure distribution on an opposing side of the valve V compared to the pledget 40. The implant clips 30 may provide a more consistent or even seal between the valve V and the tissue T by distributing pressure caused by the force of the tensioned sutures 12, 14. Further, the suture assemblies 10 may be knotless, reducing conventional knots and crimps for securing sutures 12, 14 under tension. The knotless sutures 12, 14 may be re-tensioned, which can increase alignment between the valve V and the tissue T. Additional benefits or advantages would be realized and/or achieved.

The device disclosed herein is further summarized in the following paragraphs and is further characterized by combinations of any and all various aspects described herein.

According to an aspect of the present disclosure, knotless valve suture assembly may include a pledget and a suture operably coupled with the pledget. The suture may include a first end and a second end. The suture may engage the pledget between the first end and the second end. A first predefined loop may be coupled with the first end. A second predefined loop may be coupled with the second end. A first needle may be operably coupled with the first end and the second predefined loop. A second needle may be operably coupled with the second end and the first predefined loop. At least one locking feature may be included, and the suture may extend through the at least one locking feature. The first predefined loop and the second predefined loop may be configured to be tensioned over an implant clip. The at least one locking feature may be configured to retain the first and second predefined loops under tension in response to an engagement between the at least one locking feature and the suture during a tensioning process.

According to an aspect of the present disclosure, a knotless valve suture assembly may include a pledget. A suture may extend through the pledget. The suture may include first and second ends and first and second predefined loops. Each of the first and second ends and the first and second predefined loops may be disposed proximate an abutting surface of the pledget. A first needle may be operably coupled with the first end and the second predefined loop. A second needle may be operably coupled with the second end and the first predefined loop. The first predefined loop and the second predefined loop may be configured to be tensioned over an implant clip in response to a tensioning force applied to the first and second ends.

According to an aspect of the present disclosure, a method of replacing a cardiovascular valve may include carrying a first end and a first predefined loop of a suture through tissue and a replacement valve with a first needle; carrying a second end and a second predefined loop of the suture through the tissue and the replacement valve with a second needle; positioning the first and second predefined loops to engage an implant clip; applying a tensioning force to the first and second ends; tensioning the first and second predefined loops over the implant clip; pressing the implant clip against the replacement valve with the first and second predefined loops; and locking the suture in a tensioned state with at least one locking feature.

According to an aspect of the present disclosure, a knotless valve suture assembly may include multiple sutures each including a suture including a tail end and a predefined loop. Locking features may engage the respective one of the multiple sutures. The sutures may be configured to extend through locking features, and the locking features may be configured to retain the sutures in a tensioned state. At least one needle may be coupled to each suture. Multiple implant clips may be included, including at least a first implant clip proximate to a second implant clip. The predefined loop of a first one of the multiple sutures may be configured to be tensioned over the first and second implant clips in response to a tensioning process.

According to an aspect of the present disclosure, a method of replacing a cardiovascular valve may include carrying an end and a predefined loop of a first suture through tissue and a replacement valve with at least one needle; carrying an end and a predefined loop of a second suture through the tissue and the replacement valve with at least one needle; positioning the predefined loop of the first suture around first and second implant clips; positioning the predefined loop of the second suture around the second implant clip and a third implant clip; tensioning the predefined loop of the first suture over the first and second implant clips; tensioning the predefined loop of the second suture over the second and third implant clips; pressing the first, second, and third implant clips against the replacement valve with the predefined loops of the first and second sutures in a tensioned state; locking the first suture in a tensioned state with a first locking feature; and locking the second suture in a tensioned state with a second locking feature.

According to an aspect of the present disclosure, a knotless valve suture assembly may include a first predefined suture loop, a first suture end coupled with the first predefined suture loop, a second predefined suture loop, and a second suture end coupled with the second predefined suture loop. At least one implant clip may be included. At least one of the first and second predefined suture loops may be configured to be tensioned over the at least one implant clip in response to the tensioning force applied to at least one of the first and second suture ends, respectively, to press the at least one implant clip against a replacement valve.

According to any preceding aspect of the present disclosure, at least one locking feature may include a first locking feature operably coupled with a first predefined loop and a second locking feature operably coupled with a second predefined loop.

According to any preceding aspect of the present disclosure, an implant clip may be at least one of a slotted ring, a notched clip, a C-shaped clip, and an S-shaped clip.

According to any preceding aspect of the present disclosure, a first needle may be operably coupled to a second predefined loop via a first connecting suture. A second needle may be operably coupled to a first predefined loop via a second connecting suture.

According to any preceding aspect of the present disclosure, at least one locking feature may be a spliced segment of a suture coupled with a pledget.

According to any preceding aspect of the present disclosure, a suture may include a thickened portion having stuffing proximate to a spliced segment. The stuffing may be configured to bunch during a tensioning process to lock the suture under tension.

According to any preceding aspect of the present disclosure, a suture may extend through a pledget between a first end and a first predefined loop, between the first predefined loop and a second predefined loop, and between the second predefined loop and a second end.

According to any preceding aspect of the present disclosure, an implant clip may be a slotted ring defining an insertion slot for receiving first and second predefined loops.

According to any preceding aspect of the present disclosure, an implant clip may define beveled outer edges to an insertion slot.

According to any preceding aspect of the present disclosure, an implant clip may be constructed of polyetheretherketone.

According to any preceding aspect of the present disclosure, a first end may be coupled with a first predefined loop. A suture may extend through a pledget between the first end and the first predefined loop.

According to any preceding aspect of the present disclosure, a first predefined loop may be coupled with a second predefined loop. A suture may extend through a pledget between the first predefined loop and the second predefined loop.

According to any preceding aspect of the present disclosure, a second end may be coupled with a second predefined loop. A suture may extend through a pledget between the second end and the second predefined loop.

According to any preceding aspect of the present disclosure, a suture may be configured to extend through a locking feature. The locking feature may be configured to retain the suture under tension in response to a tensioning force.

According to any preceding aspect of the present disclosure, a locking feature may be a spliced segment of a suture.

According to any preceding aspect of the present disclosure, a suture may include a thickened portion proximate to a spliced segment. The thickened portion may be configured to bunch to lock the suture in a tensioned state in response to a tensioning force.

According to any preceding aspect of the present disclosure, an implant clip may be a slotted ring defining an insertion slot for receiving first and second predefined loops.

According to any preceding aspect of the present disclosure, a step of applying a tensioning force may include reducing a size of a second predefined loop in response to applying the tensioning force to a first end and reducing a size of a first predefined loop in response to applying the tensioning force to a second end.

According to any preceding aspect of the present disclosure, at least one locking feature may include a spliced segment. A step of locking a suture includes drawing the suture through the spliced segment and forming a locking trap with the spliced segment and stuffing in the suture.

According to any preceding aspect of the present disclosure, a step of pressing an implant clip against a replacement valve may include forming a seal between the replacement valve and a tissue.

According to any preceding aspect of the present disclosure, multiple implant clips may include a third implant clip. A predefined loop of a second one of multiple sutures may be configured to be tensioned over second and third implant clips in response to a tensioning process.

According to any preceding aspect of the present disclosure, adjacent implant clips of multiple implant clips may be coupled together via sutures to form an implant ring.

According to any preceding aspect of the present disclosure, each suture may include a fixed end coupled to a respective pledget via a base knot.

According to any preceding aspect of the present disclosure, multiple implant clips may be configured as at least one of a slotted ring, a notched clip, a C-shaped clip, and an S-shaped clip.

According to any preceding aspect of the present disclosure, locking features may be spliced segments of sutures. The sutures may each include a thickened portion configured to bunch in response to the tensioning process.

According to any preceding aspect of the present disclosure, locking features may be grommets.

According to any preceding aspect of the present disclosure, a grommet may include angled inner barbs for allowing movement of a respective suture through the grommet in a single direction.

According to any preceding aspect of the present disclosure, at least one needle may include a first needle coupled to the tail end and a second needle coupled to the predefined loop via a connecting suture.

According to any preceding aspect of the present disclosure, a step of carrying an end and a predefined loop of a first suture may include carrying the end of the first suture through a replacement valve and tissue via a first needle and carrying the predefined loop of the first suture through the replacement valve and the tissue via a second needle.

According to any preceding aspect of the present disclosure, a step of carrying an end and a predefined loop of a second suture may include carrying the end of the second suture through a replacement valve and tissue via a third needle and carrying the predefined loop of the second suture through the replacement valve and the tissue via a fourth needle.

According to any preceding aspect of the present disclosure, a step of locking a first suture may include drawing the first suture through a first locking feature. The first locking feature is a spliced segment of the first suture.

According to any preceding aspect of the present disclosure, a step of locking a first suture may include drawing the first suture through the first locking feature. The first locking feature is a grommet.

According to any preceding aspect of the present disclosure, each of first and second predefined suture loops may be configured to be tensioned over at least one implant clip.

According to any preceding aspect of the present disclosure, at least one implant clip may include a first implant clip and a second implant clip.

According to any preceding aspect of the present disclosure, a first predefined suture loop may be configured to be tensioned over both a first implant clip and a second implant clip.

According to any preceding aspect of the present disclosure, a first predefined suture loop may be configured to be tensioned over a first implant clip. A second predefined suture loop may be configured to be tensioned over a second implant clip.

According to any preceding aspect of the present disclosure, a first suture may include a first suture end and a first predefined suture loop. A second suture may include a second suture end and a second predefined suture loop.

According to any preceding aspect of the present disclosure, a first pledget may be coupled with a first suture. A second pledget may be coupled with a second suture.

According to any preceding aspect of the present disclosure, a pledget may be coupled with first and second sutures.

According to any preceding aspect of the present disclosure, a first suture may be configured to extend through a first locking feature. The first locking feature may be configured to engage the first suture to retain the first suture in a tensioned state in response to a tensioning process. A second suture may be configured to extend through a second locking feature. The second locking feature may be configured to engage the second suture to retain the second suture in a tensioned state in response to the tensioning process.

According to any preceding aspect of the present disclosure, first and second locking features may be spliced segments of first and second sutures.

According to any preceding aspect of the present disclosure, first and second locking features may be grommets.

According to any preceding aspect of the present disclosure, a suture may include each of a first suture end, a first predefined suture loop, a second suture end, and a second predefined suture loop.

According to any preceding aspect of the present disclosure, a pledget may be coupled with a suture.

According to any preceding aspect of the present disclosure, a suture may be configured to extend through a locking feature, and the locking feature may be configured to engage the suture to retain the suture in a tensioned state in response to a tensioning process.

According to any preceding aspect of the present disclosure, at least one implant clip may be at least one of a slotted ring, a notched clip, a C-shaped clip, and an S-shaped clip.

According to any preceding aspect of the present disclosure, a first needle may be operably coupled with a first suture end and a first predefined suture loop to carry the first suture end and the first predefined suture loop through tissue and a replacement valve. A second needle may be operably coupled with a second suture end and a second predefined suture loop to carry the second suture end and the second predefined suture loop through the tissue and the replacement valve.

According to any preceding aspect of the present disclosure, a first needle may be operably coupled with a first suture end to carry the first suture end through tissue and a replacement valve. A second needle may be operably coupled with a first predefined suture loop to carry the first predefined suture loop through the tissue and the replacement valve. A third needle may be operably coupled with a second suture end to carry the second suture end through the tissue and the replacement valve. A fourth needle may be operably coupled with a second predefined suture loop to carry the second predefined suture loop through the tissue and the replacement valve.

According to any preceding aspect of the present disclosure, a first suture end may extend into a first predefined suture loop, the first predefined suture loop may extend into a second predefined suture loop, and the second predefined suture loop may extend into a second suture end.

According to any preceding aspect of the present disclosure, a pledget may be coupled with a suture. A first suture end and a second predefined loop may be spaced from the second suture end and the first predefined suture loop by the pledget.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A knotless valve suture assembly, comprising:
a first predefined suture loop;
a first suture end extending from the first predefined suture loop;
a second predefined suture loop;
a second suture end extending from the second predefined suture loop;
a first needle operably coupled with the first predefined suture loop; and
a second needle operably coupled with the second predefined suture loop, the first and second needles configured to carry the first and second predefined suture loops through tissue and a replacement valve, respectively; and
at least one implant clip, wherein at least one of the first and second predefined suture loops is configured to be tensioned over the at least one implant clip in response to a tensioning force applied to at least one of the first and second suture ends, respectively, to press the at least one implant clip against the replacement valve.

2. The knotless valve suture assembly of claim 1, wherein each of the first and second predefined suture loops is configured to be tensioned over the at least one implant clip.

3. The knotless valve suture assembly of claim 1, wherein the at least one implant clip includes a first implant clip and a second implant clip, and wherein the first predefined suture loop is configured to be tensioned over both the first implant clip and the second implant clip.

4. The knotless valve suture assembly of claim 1, further comprising:
a first suture including the first suture end and the first predefined suture loop; and
a second suture including the second suture end and the second predefined suture loop.

5. The knotless valve suture assembly of claim 4, further comprising:
a third needle operably coupled with the first suture end to carry the first suture end through the tissue and the replacement valve; and
a fourth needle operably coupled with the second suture end to carry the second suture end through the tissue and the replacement valve.

6. The knotless valve suture assembly of claim 4, further comprising:
a pledget, wherein the first suture extends through the pledget between the first end and the first predefined loop.

7. The knotless valve suture assembly of claim 4, further comprising:
a first locking feature through which the first suture is configured to extend, wherein the first locking feature is configured to engage the first suture to retain the first suture in a tensioned state in response to a tensioning process; and
a second locking feature through which the second suture is configured to extend, and wherein the second locking feature is configured to engage the second suture to retain the second suture in a tensioned state in response to the tensioning process.

8. The knotless valve suture assembly of claim 7, wherein the first and second locking features are at least one of spliced segments of the first and second sutures, respectively and grommets.

9. The knotless valve suture assembly of claim 1, further comprising:
a suture including each of the first suture end, the first predefined suture loop, the second suture end, and the second predefined suture loop.

10. The knotless valve suture assembly of claim 9, wherein the first suture end extends into the first predefined suture loop, wherein the first predefined suture loop extends into the second predefined suture loop, and further wherein the second predefined suture loop extends into the second suture end.

11. The knotless valve suture assembly of claim 10, further comprising:
a pledget coupled with the suture, wherein the first suture end and the second predefined loop are spaced from the second suture end and the first predefined suture loop by the pledget.

12. The knotless valve suture assembly of claim 9, further comprising:
a locking feature through which the suture is configured to extend, and wherein the locking feature is configured to engage the suture to retain the suture in a tensioned state in response to a tensioning process.

13. The knotless valve suture assembly of claim 9, wherein the first needle is operably coupled with the second suture end to carry the second suture end and the first predefined suture loop through the tissue and the replacement valve together, and wherein the second needle is operably coupled with the first suture end and the second predefined suture loop to carry the second suture end and the second predefined suture loop through the tissue and the replacement valve together.

14. The knotless valve suture assembly of claim 1, wherein the at least one implant clip is at least one of a slotted ring, a notched clip, a C-shaped clip, and an S-shaped clip.

15. A knotless valve suture assembly, comprising:
a pledget;
a suture operably coupled with the pledget, wherein the suture includes:
a first end;
a second end, wherein the suture engages the pledget between the first end and the second end;
a first predefined loop coupled with the first end; and
a second predefined loop coupled with the second end;
a first needle operably coupled with the first end and the second predefined loop;
a second needle operably coupled with the second end and the first predefined loop;
at least one locking feature, wherein the suture extends through the at least one locking feature; and
an implant clip, wherein the first predefined loop and the second predefined loop are configured to be tensioned over the implant clip, and wherein the at least one locking feature is configured to retain the first and second predefined loops under tension in response to an engagement between the at least one locking feature and the suture during a tensioning process.

16. The knotless valve suture assembly of claim 15, wherein the at least one locking feature includes:
a first locking feature operably coupled with the first predefined loop; and
a second locking feature operably coupled with the second predefined loop.

17. The knotless valve suture assembly of claim 15, wherein the at least one locking feature is a spliced segment of the suture coupled with the pledget, and wherein the suture includes a thickened portion having stuffing proximate to the spliced segment, the stuffing configured to bunch during the tensioning process to lock the suture under tension.

18. The knotless valve suture assembly of claim 15, wherein the suture extends through the pledget between the first end and the first predefined loop, between the first predefined loop and the second predefined loop, and between the second predefined loop and the second end.

19. The knotless valve suture assembly of claim 15, wherein the implant clip is a slotted ring defining an insertion slot for receiving the first and second predefined loops, and wherein the implant clip defines beveled outer edges to the insertion slot.

20. A knotless valve suture assembly, comprising:
multiple sutures each including a tail end and a predefined loop;
locking features each engaging a respective one of the multiple sutures, wherein the multiple sutures are configured to extend through the locking features, respectively, and wherein the locking features are configured to retain the multiple sutures in a tensioned state based on an engagement between the locking features and the multiple sutures, respectively; and
at least one needle coupled to each suture; and
multiple implant clips including at least a first implant clip proximate to a second implant clip, wherein the predefined loop of a first one of the multiple sutures is configured to be tensioned over the first and second implant clips.
